(19) United States
(12) Patent Application Publication
(10) Pub. No.: US 2017/0177834 A1
(43) Pub. Date: Jun. 22, 2017

(54) NANOMEDICINE OPTIMIZATION WITH FEEDBACK SYSTEM CONTROL

(71) Applicant: THE REGENTS OF THE UNIVERSITY OF CALIFORNIA, Oakland, CA (US)

(72) Inventors: Chih-Ming HO, Los Angeles, CA (US); Dean HO, Los Angeles, CA (US); Hann WANG, Los Angeles, CA (US); Dong-Keun LEE, Los Angeles, CA (US); Kai-Yu CHEN, Los Angeles, CA (US)

(73) Assignee: THE REGENTS OF THE UNIVERSITY OF CALIFORNIA, Oakland, CA (US)

(21) Appl. No.: 15/127,634

(22) PCT Filed: Mar. 23, 2015
(86) PCT No.: PCT/US2015/022056

§ 371 Date: Sep. 20, 2016

Related U.S. Application Data
(60) Provisional application No. 61/968,769, filed on Mar. 21, 2014.
(60) Provisional application No. 62/116,290, filed on Feb. 13, 2015.

(51) Int. Cl. G06F 19/00; G01N 33/50; G06N 7/08

(52) U.S. Cl. CPC G06F 19/704; G06N 7/08; G01N 33/502

(57) ABSTRACT

A cost function is specified to optimize a combination of N drugs, where the cost function includes at least one phenotypic contribution corresponding to efficacy and at least one phenotypic contribution corresponding to safety, and at least one of the N drugs is a nanomaterial-modified drug, with N being 2 or more. In vitro or in vivo tests are conducted by applying varying combinations of dosages of the N drugs to determine the phenotypic contributions from results of the tests. The results of the tests are fitted into a representation of the cost function, and, using the representation of the cost function, at least one optimized combination of dosages of the N drugs is identified.

### CROSS-REFERENCE TO RELATED APPLICATIONS

This application claims the benefit of U.S. Provisional Application Ser. No. 61/968,769, filed on Mar. 21, 2014, and the benefit of U.S. Provisional Application Ser. No. 62/116,290, filed on Feb. 13, 2015, the disclosures of which are incorporated herein by reference in their entirety.

### STATEMENT REGARDING FEDERALLY SPONSORED RESEARCH OR DEVELOPMENT

This invention was made with Government support under CMMI-0856492, CMMI-1350197, and DMR-1343991, awarded by the National Science Foundation. The Government has certain rights in the invention.

### TECHNICAL FIELD

This disclosure generally relates to nanomedicine optimization and, more particularly, to optimization of nanomaterial-modified drug combinations.

### BACKGROUND

At present, drug combinations to treat various types of diseases are often developed arbitrarily, or in an additive fashion. It can be quite challenging to simultaneously account for improved efficacy and reduced toxicity in the design of drug combinations. This is because a parameter space (e.g., a number of drugs and desired responses) can be too large to practically manage and can be too cost prohibitive for testing—6 drugs at 10 different concentrations or doses would involve at least 1 million tests. Furthermore, even modifying one drug concentration can result in too many downstream changes to accurately determine a resulting impact on any subsequent attempts to arrive at an optimal combination.

In many cases, issues with drug resistance, drug metabolism, and drug instability, and specifications to shield toxicity and for increased circulatory half-life specify that a carrier should be used for drugs. In such cases, nanomaterial-modified therapeutics have been shown to increase intratumoral retention, reduce side effects, increase half-life, and other benefits. However, current nanomedicine combination strategies are also arbitrary or additive. It can be quite challenging to rationally design a nanomedicine combination that properly optimizes for all desired responses.

It is against this background that a need arose to develop the nanomedicine optimization technique described herein.

### SUMMARY

In some embodiments, a method includes: (1) specifying a cost function to optimize a combination of N drugs, the cost function including at least one phenotypic contribution corresponding to efficacy and at least one phenotypic contribution corresponding to safety, at least one of the N drugs is a nanomaterial-modified drug, with N being 2 or more; (2) conducting in vitro or in vivo tests by applying varying combinations of dosages of the N drugs to determine the phenotypic contributions from results of the tests; (3) fitting the results of the tests into a representation of the cost function; and (4) using the representation of the cost function, identifying at least one optimized combination of dosages of the N drugs.

In other embodiments, a method includes: (1) evaluating a pool of P drugs to identify multiple optimized subsets of the P drugs having respective values of a therapeutic outcome; (2) ranking the optimized subsets according to their respective values of the therapeutic outcome; (3) selecting an optimized subset from the ranked optimized subsets, the selected optimized subset being a combination of N drugs, with N <P; (4) modifying at least one of the N drugs with a nanomaterial to provide a nanomaterial-modified combination of the N drugs; and (5) evaluating the nanomaterial-modified combination of the N drugs to identify an optimized combination of dosages of the N drugs.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

For a better understanding of the nature and objects of some embodiments of this disclosure, reference should be made to the following detailed description taken in conjunction with the accompanying drawings.

FIG. 1: An example of identifying an optimized dose with 3 tests according to an embodiment of this disclosure.

FIG. 2: A processing unit implemented in accordance with an embodiment of this disclosure.

FIG. 3: The Framework of Feedback System Control (FSC). Step 1: Drug Selection/Design—Doxorubicin (DOX), Mitoxantrone (MTX), and Bleomycin (BLEO) were loaded onto nanodiamonds (NDs) by physisoprtion, forming stable and uniform colloidal solutions, and combinations were designed. Latin hypercube sampling was applied on ND-DOX, ND-BLEO, ND-MTX, and paclitaxel (PAC) to generate 57 combinations of diverse dosing regimens. Step 2: The 57 combinations were applied to 3 types of cancer cells and 3 types of control cells by customized liquid handling robotic procedures. The viability measurements of 3 cancer cell lines and 3 control cell lines were fed into an informatics system. Step 3: The informatics system generated cellular response surfaces by regression analysis with a customized statistical model on the 57 combinations, which were triplicated and generated for a total of 171 data points for each cell line. Global combinatorial optimization was carried out by differential evolution on a surface of a therapeutic window. The predicted optimum and randomized combinations were then experimentally verified to ensure mapping accuracy, and the verified global optimum was obtained for further investigation.

FIG. 4: Nanodiamond-drug synthesis and characterization. (A) Fourier transform infrared spectroscopy (FTIR) spectra of (a) ND, (b) DOX, (c) MTX, (d) BLEO, (e) ND-DOX, (f) ND-MTX, and (g) ND-BLEO. (B) Dynamic Light Scattering analysis for the ND and ND-drugs. The graph showed the diameter of ND, ND-DOX, ND-MTX, and ND-BLEO are 46.6±0.17 nm, 120.0±0.93 nm, 109.2±0.58 nm, and 105.1±0.53 nm (n=5), and zeta potentials of ND, ND-DOX, ND-MTX, and ND-BLEO are 55.8±0.37 mV, 45.3±0.51 mV, 47.8±0.66 mV, and 52.0±0.35 mV (n=3), respectively. (C) Drug concentrations on the NDs in 1 mL of ND-drugs solution. Drug concentrations of DOX, MTX, and BLEO in each 1 mL of ND-drugs are 876±2.8 μg, 987±1.0 μg, and 329±1.0 μg, respectively (n=3).

FIG. 5: The formation of the therapeutic window surface from the cellular response surfaces. (A) A response surface showing the cellular response of the MCF10A (control) cells to varying combinations of ND-DOX, ND-BLEO, ND-MTX, and PAC. The concentrations of ND-BLEO and PAC were fixed at optimal dosage while the surface is plotted by varying the concentration of ND-MTX and ND-DOX. Two dimensions (concentrations) were fixed in order to project the full 5 dimensional surface in 3 dimensions (n=3). (B) A response surface showing MDA-MB-231 (triple negative breast cancer) cellular response to the ND combinations (n=3). (C) A therapeutic window surface between the MDA-MB-231 (cancer) and MCF10A (control) cells when the 4-drug combinations were applied (n=3). The concentration levels were reversed to allow a better viewing angle of the higher therapeutic window surface area. The pyramid denotes the experimentally-verified optimal therapeutic window, 51.50±3.51%, with the bottom tip of the pyramid representing experimental mean, and the height of the pyramid representing the standard error. The dosing of the optimum ND combination was ND-DOX: 9.88E-7 M, ND-BLEO: 1.12E-9 M, ND-MTX: 2.10E-5 M, and PAC: 2.02E-10 M. The FSC predicted optimum therapeutic window was located at the same concentration as the pyramid and had a value of 47.01%. A t-test generated a p-value of 0.35 (null hypothesis holds true), indicating that the FSC prediction was statistically significant to predict the experimental optimum. (D) A response surface of H9C2 (n=3). (E) A response surface of MDA-MB-231 (n=3). (F) A therapeutic window surface between H9C2 (control) and MDA-MB-231 (cancer) (n=3). The experimental optimal therapeutic window was 21.46±4.00% and located at ND-DOX: 4.88E-8 M, ND-BLEO: 2.00E-6 M, ND-MTX: 2.34E-9 M, and PAC: 2.00E-5 M; predicted optimal is 20.71% (p=0.78). (G) A response surface of IMR-90 (n=3). (H) A response surface of MDAMB-231 (n=3). (I) A therapeutic window surface between IMR-90 (control) and MDA-MB-231 (cancer) (n=3). The experimental optimal therapeutic window was 15.80±6.19% and located at ND-DOX: 1.08E-8 M, ND-BLEO: 4.47E-7 M, ND-MTX: 8.93E-8 M, and PAC: 2.94E-5 M; predicted optimal is 9.53% (p-value=0.48).

FIG. 6: Pearson correlation plot for verification experiment versus model prediction. Pearson correlation plots of the FSC predicted viabilities of (A) MCF10A, (B) H9C2, (C) IMR-90, (D) MDA-MB-231, (E) MCF7, and (F) BT20, versus experimental values (n=3) of these three control cells and three cancer cells in the verifications, showing high correlation of R=0.90, R=0.93, R=0.93, R=0.93, R=0.82, and R=0.92, respectively, between predicted values and experimental values (n=3).

FIG. 7: Nanodiamond combination performance versus nanodiamond single drug administration. Bar graph showing the therapeutic window between the viabilities of the cancer cell, MDA-MB-231, and control cell, MCF10A, from the optimum dosing of each drug or drug combination in the experiment (n=3). The length of the vertical line shows the standard error of each condition. The concentration of optimal dosing is provided in the table at the bottom.

FIG. 8: Nanodiamond combination performance versus unmodified combinations. (A) Therapeutic window of the optimal ND combination versus optimal unmodified combination for MCF10A (control) and MDA-MB-231 (cancer cell) (p=0.0146, n=3); the averaged performance of unmodified and ND combination is also listed. Notice that the optimal drug concentration is different for ND-modified and unmodified combinations. (B) Therapeutic window of the optimal ND combination versus optimal unmodified combination in the case of H9C2 (control) and MDA-MB-231 (cancer cell) (p=0.0012, n=3). (C) Therapeutic window of the optimal ND combination versus optimal unmodified combination in the case of IMR-90 (control) and MDAMB-231 (cancer cell) (p=0.0301, n=3).

FIG. 9: Nanodiamond optimum combination performance versus nanodiamond randomized combinations. The therapeutic windows of a panel of cancer and control pairs are shown. Cancer cell lines include MDA-MB-231, MCF7, and BT20, and control cells include MCF10A, H9C2, and IMR-90, forming a total of 6 possible cancer-control pairs (n=3).

FIG. 10: Standard curve of three drugs for drug loading analysis. Standard curve derived by 0 to 200 μg/mL serial concentration range of (A) DOX (n=3), (B) MTX (n=3), and (C) BLEO (n=3) for calculating the concentration of drugs onto NDs. The spectra were measured at the wavelengths of (A) 550 nm, (B) 590 nm, and (C) 290 nm.

FIG. 11: Therapeutic window of ND combinations versus single drugs. Bar graph showing the therapeutic window between the viabilities of the cancer cells, MDA-MB-231, MCF7, and BT20, and control cells, MCF10A, H9C2, and IMR-90, from the optimum dosing of each drug or ND-drug combination in the experiment (n=3). The length of the vertical line shows the standard error of each condition.

### DETAILED DESCRIPTION

### Overview

Embodiments of this disclosure circumvent many technology roadblocks encountered in optimizing complex systems in general. In some embodiments, a specific example of treating diseases of a biological system with optimized drug combinations (or combinatorial drugs) and respective doses or concentrations is used to illustrate certain aspects of this disclosure. The goal of optimization of some embodiments of this disclosure can be any one or any combination of reducing labor, reducing cost, reducing risk, increasing reliability, increasing efficacies, reducing side effects, reducing toxicities, and alleviating drug resistance, among others. A biological system can include, for example, an individual cell, a collection of cells such as a cell culture or a cell line, an organ, a tissue, or a multi-cellular organism such as an animal (e.g., a pet or a livestock), an individual human patient, or a group of human patients (e.g., a population or sub-population of human patients). A biological system can also include, for example, a multi-tissue system such as the nervous system, immune system, or cardio-vascular system. More generally, embodiments of this disclosure can also optimize wide varieties of other complex systems by applying pharmaceutical, chemical, nutritional, physical, or other types of stimulations. Applications of embodiments of this disclosure include, for example, optimization of drug combinations, vaccine or vaccine combinations, chemical synthesis, combinatorial chemistry, drug screening, treatment therapy, cosmetics, fragrances, and tissue engineering, as well as other scenarios where a group of optimized stimulations is of interest.

Stimulations (or other system inputs) can be therapeutic stimuli to treat diseases, control immunosuppression, or otherwise promote improved health, such as pharmaceutical (e.g., single drug or combinatorial drugs, including existing, generic, and later developed drugs, which are applied towards existing therapeutics, repurposing, or later developed drug optimization), biological (e.g., protein therapeutics, antibody therapeutics, peptide-based therapeutics, hormones, inhibitors, DNA, RNA, or other nucleic acid therapeutics, and immunotherapeutic agents, such as cytokines, chemokines, and immune effector cells such as lymphocytes, macrophages, dendritic cells, natural killer cells, and cytotoxic T lymphocytes), chemical (e.g., chemical compounds, metal-based compounds, ionic agents, and naturally-derived compounds, such as traditional eastern medicine compounds), and physical (e.g., light, heat, electrical stimuli, such as electrical current or pulse, and mechanical stimuli, such as pressure, shear force, or thermal energy), among others. Imaging agents can be considered as drugs in some embodiments, and these agents can be optimized as well. Examples of imaging agents include magnetic resonance imaging (MRI) contrast agents (e.g., gadolinium-based, magnesium sulfate-based, and iron oxide-based, among others), computed tomography (CT) agents, computed axial tomography (CAT) agents, positron emission tomography (PET) agents, near-infrared agents, fluorescent agents, nanomaterial-based agents, glucose, and barium-based agents, among others. Optimization of immunotherapy or chemotherapy regimens are encompassed by this disclosure, such as T-cell immunotherapy (e.g., Chimeric Antigen Receptor (CAR) T-cell therapy and Cytotoxic T Lymphocytes (CTL)) and protein and protein fragment-based immunotherapy, among others, with optimized combinations to either promote or sustain T-cell activation against cancer. Furthermore, along with immunotherapy or chemotherapy regimens, rapid optimization of drug therapy in concert with such regimens can be attained as well. For example, T-cell immunotherapy with optimized drug combinations can be applied to optimize therapeutic efficacy and safety. In addition, T-cell immunotherapy with optimized combinations of various compounds can be used to optimize T-cell activation to improve treatment efficacy and safety. Moreover, veterinary therapeutic agents can be optimized in some embodiments.

In the case of drugs, for example, drug release can be administered systemically via any one or any combination of intravenous, oral, intramuscular, intraperitoneal, via eye drops, transdermal, via ointments/creams, and via a medical device (e.g., pump infusion, implantable, transdermal, ocular, and so forth).

Diseases can include, for example, cancer, cardiovascular diseases, pulmonary diseases, atherosclerosis, diabetes, metabolic disorders, genetic diseases, viral diseases (e.g., human immunodeficiency virus, hepatitis B virus, hepatitis C virus, and herpes simplex virus-1 infections), bacterial diseases, and fungal diseases, among others, which can be drug-sensitive or drug-resistant. Some embodiments of this disclosure are implemented and validated in multiple cancer cell lines for efficacy and multiple control cell lines for toxicity, but the optimization technique can be expanded towards other disorders and health related applications, such as other cancers, infectious diseases, nutraceuticals, herbal, or eastern medication, homeopathic treatment, cosmetics, immunotherapy and immunomodulation, and probiotic optimization, among others. More generally, the optimization technique of embodiments of this disclosure is applicable towards virtually all classes of diseases, since the diseases mediate phenotypic change which is an output that the optimization technique uses to realize optimal therapeutic outcomes. Optimization can include complete optimization in some embodiments, but also can include substantially complete or partial optimization in other embodiments.

Some embodiments of this disclosure leverage a Feedback System Control (FSC) platform, which allows integration of experimental and analytical modalities, such as differential evolution and Gur Game, among others. The FSC platform can account for relevant biological interactions, including, for example, intracellular signaling pathway processes, linear and non-linear interactions, intermolecular interactions, intercellular interactions, and genotypic interactions and processes, because the FSC platform is phenotypically-driven, which serves as a foundation for personalized medicine.

In some embodiments, a FSC-directed nanomedicine combination can optimize therapeutic efficacy while allowing a reduced drug dosing to be engineered into the combination, and a truly optimized outcome can be realized in a rapid fashion to mediate phenotypically-driven nanomedicine treatments. Furthermore, the technique can be used to formulate combinations of different types of nanomaterial platforms (e.g., different types of nanoparticles), combinations of drugs using the same type of nanomaterial platform (e.g., same type of nanoparticles), or combinations of nanomaterial-modified drugs and unmodified drugs together. This represents a powerful approach towards the rational design of nanomedicine combination strategies for optimized safety and efficacy and with reduced labor and costs.

### Nanomedicine Optimization

Stimulations can be applied to direct a complex system towards a desired state, such as applying drugs to treat a patient. The types and the amplitudes (e.g., dosages) of applying these stimulations are part of input parameters that can affect the efficiency in bringing the system towards the desired state. However, N types of different drugs with M different dosages for each drug will result in M^{N} possible drug-dosage combinations. To identify an optimized or even near optimized combination by multiple tests on all possible combinations is prohibitive in practice. For example, it is not practical to perform all the possible drug-dosage combinations in in vitro or in vivo tests for finding an effective drug-dosage combination as the number of drugs and dosages increase. The inclusion of nanomaterial platforms can add further dimensions to the input parameter space, further complicating the optimization of nanomaterial-modified drug combinations.

Some embodiments of this disclosure provide a technique that allows a rapid search for optimized combinations of stimulations to guide complex systems towards their desired states. An optimization technique can be used to identify at least a subset, or all, optimized combinations or sub-combinations of stimulations that produce desired states of a complex system. Taking the case of combinational drugs, for example, a pool of P different drugs can be evaluated to rapidly identify an optimized combination of N different drugs and optimized dosages of the N drugs, where the N drugs are a subset of the pool of P drugs, namely P is greater than N, and N is greater than 1, such as 2 or more, 3 or more, 4 or more, 5 or more, 6 or more, 7 or more, 8 or more, 9 or more, or 10 or more. The optimization technique also can be used to optimize a single drug administration, such that N, more generally, can be 1 or greater than 1. In some embodiments, at least one of the N drugs is a nanomaterial-modified drug, and the N drugs and one or more nanomaterial platforms can be evaluated to rapidly identify optimized drug-nanomaterial-dosage combinations. For example, the technique can be used to identify optimized combinations of drugs modified with different types of nanomaterial platforms (e.g., different types of nanoparticles), combinations of drugs modified with the same type of nanomaterial platform (e.g., same type of nanoparticles), or combinations of nanomaterial-modified drugs and unmodified drugs together. Thus, for example, the technique can be used to identify optimized combinations of multiple drugs that are loaded onto one nanomaterial platform (e.g., a nanoparticle loaded with multiple drugs), or multiple nanomaterial platforms that are each loaded with one or more drugs for parallel administration.

In some embodiments, a pool of P different drugs can be evaluated according to a FSC optimization technique to identify at least one combination of N different candidate drugs for further evaluation. In some embodiments, the candidate drugs are unmodified drugs at this first stage of FSC optimization. Next, the combination of candidate drugs, optionally supplemented or replaced with one or more other candidate drugs, are subjected to a second stage of FSC optimization. Specifically, one or more of the candidate drugs are nanomaterial-modified, and the combination of candidate drugs (including at least one nanomaterial-modified drug) is evaluated according to the FSC optimization technique to identify optimized dosages of the candidate drugs in the combination. By applying such two-stage FSC optimization, a large pool of unmodified drugs can be evaluated to identify a subset of drugs for further evaluation, and optimized dosages of this subset of drugs (at least one of which is nanomaterial-modified) can be identified. Evaluation can be made of drugs indicated from the first stage of optimization, where safety or efficacy readouts can be used based on defined criteria (e.g., based on minimum levels of efficacy or safety), to determine which drugs might have been rejected initially, and which can be re-entered into a combination of candidate drugs and then modified with a nanomaterial platform, which will render the re-entered drugs more effective or less toxic, allowing their addition to a final, optimized combination.

For a first stage of optimization, it is contemplated that identification of candidate combinations of unmodified drugs can be carried out by performing retrospective analysis of in vitro or in vivo data, using FSC optimization to derive a phenotypic response surface to identify optimized drug combinations. From analysis of this retrospective data to identify a candidate combination, one or more drugs of the combination can be modified with nanomaterials to allow further optimization. This would be applicable towards populations/groups of cells and pre-clinical data, or data over the course of time. Databases of how biological systems (cellular to higher level) respond to varying iterations of drug combinations can be compiled, and a retrospective FSC analysis can identify optimized combinations for subsequent further evaluation with nanomaterials.

Examples of nanomaterial platforms that can be included in rationally-designed, optimized combinations include nanodiamonds, as well as others that are Food and Drug Administration (FDA)-approved (e.g., liposomal-modified and albumin-modified) or in clinical trials (e.g., poly(lactic-co-glycolic acid) (PLGA) nanoparticles, gold nanoshells, cyclodextrin nanoparticles, and other polymeric or metallic nanoparticles). These platforms can also include proteins, protein fragments, nucleic acids, aptamers, antibodies, sugars or carbohydrates, and other modification groups that can enhance the efficacy, safety, circulatory half-life, or other relevant therapeutic properties. As used herein, the term “nanomaterial” refers to an object that has at least one dimension in the nanometer (nm) range of about 1 nm to about 1 micrometer (μm), or greater than about 1 μm in the case of clusters of nanomaterials. The nm range includes the “lower nm range,” which refers to a range of dimensions from about 1 nm to about 10 nm, the “middle nm range,” which refers to a range of dimensions from about 10 nm to about 100 nm, and the “upper nm range,” which refers to a range of dimensions from about 100 nm to about 1 μm. In the case of certain modification groups such as sugars, carbohydrates, nucleic acids, protein fragments, or other similar structures, the dimensions of these structures may reside in the near sub-nanometer range, such as about 2-9 angstroms. A nanomaterial can have any of a wide variety of shapes and aspect ratios (e.g., about 1 to about 3, or greater than about 3), and can be formed of a wide variety of materials, such as lipids, proteins, oligosaccharides, metals, metal alloys, polymers, semiconductors, carbon, silicon, and ceramics, among others. Examples of additional nanomaterials include nanowires, nanotubes, nanoshells, nanoparticles, and other nanostructures. Modification of a drug using a nanomaterial can occur in a variety of ways, such as conjugation, absorption, adsorption, and enclosure within the nanomaterial, among others.

In some embodiments, the FSC technique for optimization of combinatorial drugs can be carried out in two stages as follows.

First Stage

In a first stage, a pool of P different drugs is evaluated according to the FSC optimization technique to identify at least one combination of N different candidate drugs for further evaluation. The pool of P drugs, of which the N candidate drugs are a subset, are unmodified drugs at the first stage of FSC optimization. For example, P is greater than 1, such as 5 or more, 10 or more, 15 or more, 20 or more, 25 or more, 30 or more, 35 or more, 40 or more, 45 or more, or 50 or more.

A cost function h is specified for a complex biological system being evaluated. Taking the case of combinational drugs tested on cell cultures, for example, the cost function can be a function of X, which is a vector of input parameters in an input parameter space (e.g., a combination of dosages of drugs sampled according to an experimental design methodology), and can be specified as a therapeutic window based on a viability of healthy control cells subjected to X and a viability of diseased (e.g., cancerous or tumor) cells subjected to X, where the former corresponds to safety of X, and the latter corresponds to efficacy of X. Other cost functions can be defined, such as including an interaction effect among drugs of X, to account for whether the drugs interact synergistically, antagonistically, or when the effect of the drugs is additive. The cost function h represents an overall therapeutic outcome or response to be optimized (e.g., enhanced or maximized), and includes a combination (e.g., a weighted sum) of phenotypic contributions or responses, including safety or toxicity when X is applied to healthy control cells, and efficacy when X is applied to diseased cells. The cost function h can be represented as a response surface that is a function of input parameters within a multi-dimensional input parameter space. Other relevant phenotypic contributions can be used in the cost function h by applying proper transformations to adjust a range and scale of the phenotypic contributions, such as those related to improved tolerance, enlarged therapeutic window, reduced drug dosages, and broad reduction of side effects. Certain phenotypic responses are desirable, such as drug efficacy or drug safety, while other phenotypic responses are undesirable, such as drug toxicity or drug side effects. In the case of the latter phenotypic responses, their weighting factors serve as penalty factors in the optimization of combinatorial drugs. Specific examples of other phenotypic contributions include cell death, cytotoxicity (e.g., using lactate dehydrogenase (LDH) assays), genetic modification (e.g., using Hpall tiny fragment Enrichment by Ligation-mediated PCR assays), caspase levels, gene expression levels, protein expression levels, and epigenetic signal and post translational modifications, among others.

In some embodiments, a response of a complex system to multiple inputs can be represented by a low order function, such as a second order (or quadratic) function, although a first order (or linear) function as well as a third order (or cubic) function are also contemplated as possible low order equations. Also, higher order functions are contemplated for other embodiments. Taking the case of combinational drugs, for example, an overall therapeutic response (as represented by the cost function h) can be specified as a function of drug dosages as follows:

$$h = {h}_{0} + {\sum }_{i} {a}_{i} {x}_{i} + {\sum }_{{ii}^{′}} {b}_{{ii}^{′} {x}_{i} {x}_{{i}^{′}}} + higher order terms \qquad ( 1 )$$

where xᵢ is a dosage of an drug from the pool of P total drugs, h₀ is a parameter (e.g., a constant) representing a baseline response, aᵢ is a parameter (e.g., a constant) representing a single drug response coefficient, bᵢᵢ, is a parameter (e.g., a constant) representing a drug-drug interaction coefficient, and the summations run through P. If cubic and other higher order terms are omitted, then the cost function h can be represented by a quadratic function of the drug dosages xᵢ. As noted above, other representations, including third and higher order functions or the use of linear regression, are also contemplated. Also, although a specific example of combinational drugs is used, it should be noted that the above equation more generally can be used to represent a wide variety of other complex systems as a function of multiple input parameters.

For the case of P=1 (a pool of 1 drug), then:

h=h₀+a₁x₁+b₁₁x₁x₁ (2)

with a total of three constants, h₀, a₁, and b₁₁.

For the case of P=2 (a pool of 2 drugs), then:

h=h₀+a₁x₁+a₂x₂+b₁₂x₁x₁+b₂₂x₂x₂ (3)

with a total of six constants, h=h₀+a₁, a₂, b₁₂, b₁₁, and b₂₂.

More generally for P total drugs, a total number of parameters m is 1+2P+(P(P−1))/2. If one drug dosage is kept constant in the evaluation, the number of parameters m can be further reduced to 1+2(P−1)+((P−1)(P−2))/2, for P>1. Table 1 below sets forth a total number of parameters in a quadratic cost function with respect to a total number drugs in a pool of drugs being evaluated.

TABLE 1

| | | Constants (m) |
|---|---|---|
| | | (if one drug |
| | | dosage is kept |
| Drugs (P) | Constants (m) | constant) |
| 1 | 3 | — |
| 2 | 6 | 3 |
| 3 | 10 | 6 |
| 4 | 15 | 10 |
| 5 | 21 | 15 |
| 6 | 28 | 21 |

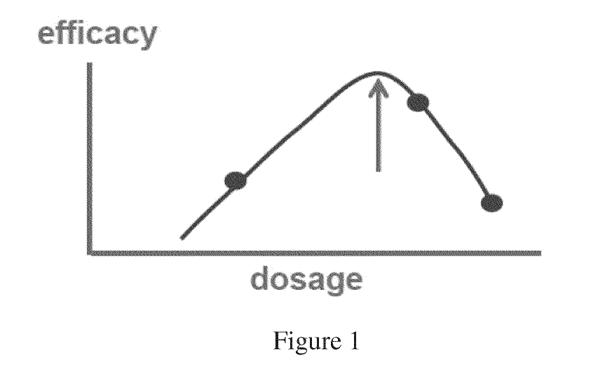

An experimental design methodology is used to guide the selection of tests to sample an input parameter space. The experimental design methodology can allow exposure of salient features of a complex system being evaluated, and can reveal a combination or sub-combination of input parameters of greater significance or impact in affecting a state of the complex system. Selection of the experimental design methodology can be according to a particular cost function of the complex system being evaluated. Examples of experimental design methodologies include Latin hypercube sampling, central composite design, d-optimal design, orthogonal array design, full factorial design, and fractional factorial design, among others. In the case of combinational drugs, for example, an experimental design methodology can be used to guide the selection of drug dosages for in vitro tests. In connection with the experimental design methodology, possible dosages can be narrowed down into a few discrete levels. FIG. 1 shows an example of the design of tests to represent an efficacy-dosage response surface. As shown in FIG. 1, the tests are designed such that at least one tested dosage lies on either side of a peak or maximum in the response surface in order to represent the surface as a quadratic function.

Once tests are designed, therapeutic outcomes (e.g., phenotypic responses) are measured by testing each combination of input parameters sampled according to the experimental design methodology, such as by applying each combination of drug dosages in vitro, in vivo, or in clinical/human tests.

Next, a representation of the cost function is fitted using values of the cost function measured or derived from the test results. Fitting of the cost function can be carried out by linear regression, Gaussian process regression, support vector machine regression, Bayesian regression, or another suitable technique. Based on the fitting performance between the test results and the fitted representation of the cost function, additional tests can be conducted to improve the accuracy of the fitted representation. Once the fitted representation with a desired accuracy is achieved, a globally or locally optimized combination of input parameters is determined or predicted using the fitted representation, such as by locating extrema using a stochastic or a deterministic optimization technique. Examples of stochastic techniques include simulated annealing, Markov chain Monte Carlo (MCMC), genetic optimization, differential evolution, and Gur game, among others. Examples of deterministic techniques include steepest descent and conjugate gradient, among others. FIG. 1 shows an example of identifying an optimized dosage of a single drug regimen with 3 tests. An optimized combination of input parameters predicted from a fitted representation can be experimentally verified, such as by applying the optimized combination in vitro, in vivo, or in clinical/human tests.

In the case of a relatively large pool of drugs being evaluated (e.g., P≧5, 10, 50, or even more), optimized sub-combinations of drugs can be identified to facilitate subsequent evaluation via nanomaterial modification. For example, in the case of a pool of 6 total drugs, all optimized sub-combinations of 2 drugs from the pool of drugs can be identified, by setting dosages of 4 drugs in the pool to zero to effectively reduce a 6 dimensional input parameter space to a 2 dimensional input parameter space, and locating maxima with respect to the 2 remaining dimensions. In this example of the pool of 6 drugs, a total of 15 different optimized sub-combinations of 2 drugs can be identified. Also, still in the case of the pool of 6 drugs, all optimized sub-combinations of 3 drugs from the pool of drugs can be identified, by setting dosages of 3 drugs in the pool to zero to effectively reduce the 6 dimensional input parameter space to a 3 dimensional input parameter space, and locating maxima with respect to the 3 remaining dimensions. In this example of the pool of 6 drugs, a total of 20 different optimized sub-combinations of 3 drugs can be identified. Also, still in the case of the pool of 6 drugs, all optimized sub-combinations of 4 drugs from the pool of drugs can be identified, by setting dosages of 2 drugs in the pool to zero to effectively reduce the 6 dimensional input parameter space to a 4 dimensional input parameter space, and locating maxima with respect to the 4 remaining dimensions. In this example of the pool of 6 drugs, a total of 15 different optimized sub-combinations of 4 drugs can be identified. Thus, by conducting as few as 28 tests for the pool of 6 drugs in a single experiment, 50 (=15+20+15) optimized sub-combinations of 2, 3, and 4 drugs can be identified as potential candidates for further evaluation via nanomaterial modification.

To facilitate further evaluation in a second stage of FSC optimization, optimized sub-combinations of drugs that are identified can be ranked according to their respective values of a therapeutic outcome when administered at their respective optimized dosages. Based on the ranking, a subset of the optimized sub-combinations that is most suitable can be selected for nanomaterial modification in the second stage of FSC optimization. Values of the therapeutic outcome used for the ranking can be those predicted from a fitted representation of a cost function, or can be those experimentally verified by testing at predicted optimized dosages. For example, in the case of a cost function specified as a therapeutic window and a pool of 6 total drugs, optimized sub-combinations of 2, 3, and 4 drugs can be ranked according to their respective values of the therapeutic window, where higher values are generally indicative of either, or both, higher efficacy and higher safety, and lower values are generally indicative of either, or both, lower efficacy and lower safety. In this example, a suitable sub-combination of N drugs can be selected for further evaluation, where N is 2, 3, or 4. It is also contemplated that multiple sub-combinations can be selected for further evaluation.

Selection of a suitable sub-combination of N drugs for further evaluation can be according to one criterion or a combination of different criteria. For example, a suitable sub-combination can be selected as the highest ranked sub-combination, or can be selected from among those having values of a therapeutic outcome at or above a threshold value or within a particular percentile range, such as within a top 50 percentile, a top 40 percentile, a top 30 percentile, a top 20 percentile, a top 10 percentile, a top 5 percentile, or a top 1 percentile. As another example, a suitable sub-combination can be selected even if ranked lower according to its value of a therapeutic outcome, such as where evaluation is made of one or more drugs in the sub-combination to determine that the drugs might have yielded a lower efficacy readout or a higher toxicity readout, but where modification of the drugs with a nanomaterial platform can render the drugs more effective or less toxic. In particular, nanomaterial modification can increase efficacy of a drug by, for example, decreased drug resistance through improved intratumoral drug retention or decreased drug flux from a tumor, and nanomaterial modification can increase safety of a drug by, for example, reducing toxicity of the drug as it is transported through a bloodstream. Thus, for example, a suitable sub-combination can be selected if a lower ranking is a result of a high efficacy being counterbalanced by a high toxicity, but where nanomaterial modification can reduce or shield against the high toxicity, or a suitable sub-combination can be selected if a lower ranking is a result of a high safety being counterbalanced by a high drug resistance, but where nanomaterial modification can reduce or shield against the high drug resistance.

Second Stage

Once a suitable sub-combination of N candidate drugs is selected from the pool of P drugs, the selected candidate drugs are subjected to a second stage of FSC optimization. Specifically, one or more of the candidate drugs are nanomaterial-modified, and the candidate drugs (including at least one nanomaterial-modified drug) is evaluated according to the FSC optimization technique to identify optimized dosages of the candidate drugs in the sub-combination. Nanomaterial modification can include modifying the candidate drugs using different and respective types of nanomaterial platforms (e.g., different types of nanoparticles), modifying the candidate drugs using the same type of nanomaterial platform (e.g., same type of nanoparticles), or using nanomaterial-modified drugs and unmodified drugs together.

Implementation of the second stage of FSC optimization can be similar to that of the first stage of FSC optimization. Thus, for example, a cost function h is specified to represent an overall therapeutic response, and is specified as a function of drug dosages as explained with reference to equations (1), (2), and (3), except where the summations run through N, and a total number of parameters in a quadratic cost function is specified in terms of N in place of P. An experimental design methodology is used to guide the selection of tests to sample a drug dosage parameter space, and, once tests are designed, therapeutic outcomes (e.g., phenotypic responses) are measured by testing each combination of drug dosages sampled according to the experimental design methodology, such as by applying each combination of drug dosages in vitro, in vivo, or in clinical/human tests. Next, a representation of the cost function is fitted using values of the cost function measured or derived from the test results, and a globally or locally optimized combination of drug dosages is determined or predicted using the fitted representation, such as by locating extrema using a stochastic or a deterministic optimization technique. An optimized combination of drug dosages predicted from the fitted representation can be experimentally verified, such as by applying the optimized combination in vitro, in vivo, or in clinical/human tests.

In some embodiments, the two-stage FSC optimization can be conducted in vitro to identify a FSC-directed nanomedicine combination, which is then selected for animal tests. A similar procedure can be conducted in moving from animal tests to clinical/human trials.

### Processing Unit

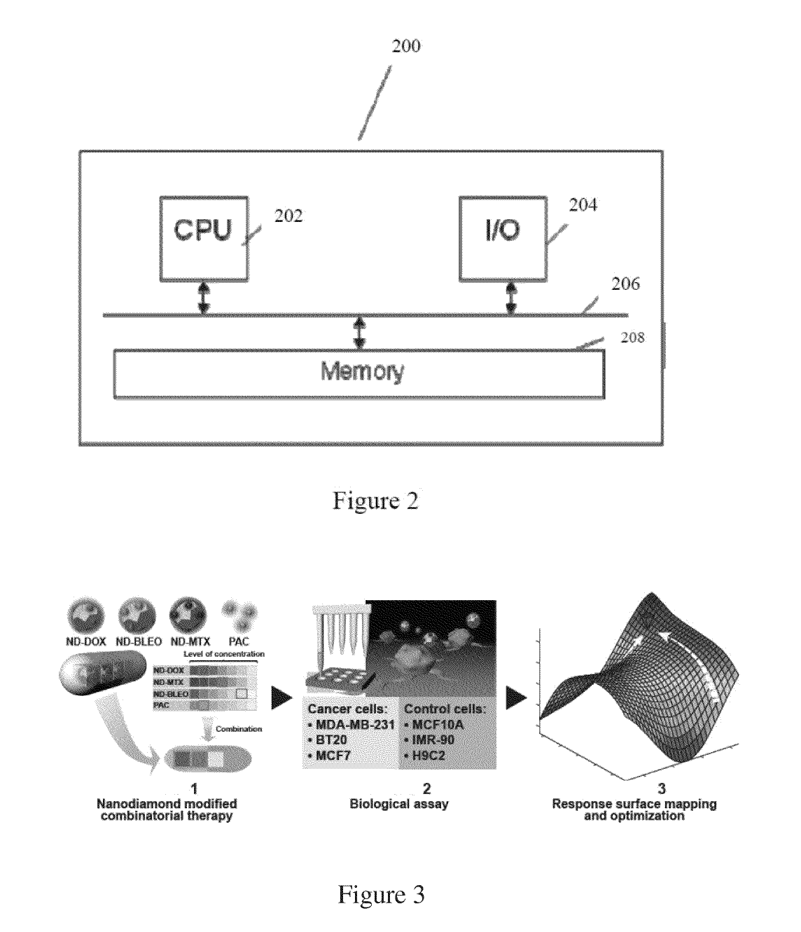

FIG. 2 shows a processing unit 200 implemented in accordance with an embodiment of this disclosure. Depending on the specific application, the processing unit 200 can be implemented as, for example, a portable electronics device, a client computer, or a server computer. Referring to FIG. 2, the processing unit 200 includes a central processing unit (CPU) 202 that is connected to a bus 206. Input/Output (I/O) devices 204 are also connected to the bus 206, and can include a keyboard, mouse, display, and the like. An executable program, which includes a set of software modules for certain operations described in this disclosure, is stored in a memory 208, which is also connected to the bus 206. The memory 208 can also store a user interface module to generate visual presentations.

An embodiment of this disclosure relates to a non-transitory computer-readable storage medium having computer code thereon for performing various computer-implemented operations. The term “computer-readable storage medium” is used herein to include any medium that is capable of storing or encoding a sequence of instructions or computer codes for performing the operations described herein. The media and computer code may be those specially designed and constructed for the purposes of this disclosure, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as floptical disks; and hardware devices that are specially configured to store and execute program code, such as application-specific integrated circuits (ASICs), programmable logic devices (PLDs), and ROM and RAM devices. Examples of computer code include machine code, such as produced by a compiler, and files containing higher-level code that are executed by a computer using an interpreter or a compiler. For example, an embodiment of this disclosure may be implemented using Java, C++, or other object-oriented programming language and development tools. Additional examples of computer code include encrypted code and compressed code. Moreover, an embodiment of this disclosure may be downloaded as a computer program product, which may be transferred from a remote computer (e.g., a server computer) to a requesting computer (e.g., a client computer or a different server computer) via a transmission channel. Another embodiment of this disclosure may be implemented in hardwired circuitry in place of, or in combination with, machine-executable software instructions.

### EXAMPLE

The following example describes specific aspects of some embodiments of this disclosure to illustrate and provide a description for those of ordinary skill in the art. The example should not be construed as limiting this disclosure, as the example merely provides specific methodology useful in understanding and practicing some embodiments of this disclosure.

### Example 1

### Mechanism-Independent Optimization of Combinatorial Nanodiamond and Unmodified Drug Delivery Using a Phenotypically-Driven Platform Technology

Overview

Combination chemotherapy can mediate drug synergy to improve treatment efficacy against a broad spectrum of cancers. However, conventional multi-drug regimens are often additively determined, which can provide good cancer-killing efficiency but can be insufficient to address the nonlinearity in dosing. Despite improved clinical outcomes by combination treatment, multi-objective combinatorial optimization, which takes into account tumor heterogeneity and balance of efficacy and toxicity, remains challenging given the sheer magnitude of the combinatorial dosing space. To enhance the properties of the therapeutic agents, the field of nanomedicine has realized drug delivery platforms that can enhance therapeutic efficacy and safety. However, optimal combination design that incorporates nanomedicine agents still faces the same hurdles as unmodified drug administration. This example reports application of a powerful phenotypically-driven platform, termed FSC, that systematically and rapidly converges upon a combination of three nanodiamond-modified drugs and one unmodified drug that is simultaneously optimized for efficacy against multiple breast cancer cell lines and safety against multiple control cell lines. Specifically, a therapeutic window achieved from an optimally efficacious and safe nanomedicine combination was markedly higher compared to that of an optimized unmodified drug combination and nanodiamond monotherapy or unmodified drug administration. The phenotypically-driven foundation of the FSC implementation does not require any cellular signaling pathway data, and innately accounts for population heterogeneity and non-linear biological processes. Therefore, FSC is a broadly-applicable platform for both nanotechnology-modified and unmodified therapeutic optimization that represents a promising path towards phenotypic personalized medicine (PPM).

Introduction

A primary focus of modern cancer drug discovery has largely relied on target and combination-based therapy. Despite improvements in treatment outcomes through angiogenic inhibition, anti-proliferation, and other targets, issues related to drug resistance, patient toxicity, and sub-optimal efficacy persist. Drug resistance is a key challenge that can inevitably limit the efficacy of targeted therapy and arises from the innate characteristics of a network of signaling pathways that behaves as a complex system. A number of systems biology studies have shown that cellular pathways form complex networks and that their collective dynamics drive phenotypic outcomes. Importantly, network dynamics cannot be readily explained by behavior of an individual component, and, as such, therapeutically addressing several elements of a diseased network is important but difficult to optimize at present. Therefore, multi-drug resistant cancer is one resulting example of a complex system characterized by system robustness, redundant pathways, cross-talk, anti-target, counter-target activity, as well as compensatory and neutralizing actions. These complex interactions often render targeted treatment ineffective in the long run.

To address these and other challenges associated with drug-resistant cancer treatment, combination therapy may enhance the systematic modulation of cellular pathways, reducing the likelihood of relapse, and showing higher selectivity against cancer cells over healthy cells. As such, combination therapy is the clinical standard for multiple cancer treatment regimens. However, conventional multi-drug regimens are often additively determined where in vitro and pre-clinical efficacious doses are combined and multiplied by a scaling factor for clinical administration. In addition, combinatorial chemotherapy regimens are often added near the maximum tolerated doses (MTD), which has been a practice for ensuring cancer-killing efficiency. Despite improvements in clinical outcomes observed following the advent of combination treatment, the multi-endpoint optimization of combinatorial therapy is a challenging target given the sheer magnitude of a parametric space when considering patient heterogeneity and other factors.

Outside of formulating regimens to deliver multiple drugs to improve treatment outcomes, modifying the drugs themselves to overcome drug resistance and reduce toxicity has been a focus of the nanomedicine field and resulted in promising findings. These approaches resulted in improved intratumoral drug retention, decreased side effects, and improved pharmacokinetic profiles. Therefore, incorporating nanotechnology-modified therapies into drug combinations may provide even further enhanced treatment outcomes.

With regards to combination drug dosing, optimal intervention of drug combinations can be dosage dependent and can be characterized by therapeutic synergism, additivity, or antagonism. Certain methods have found utility associated with combination dosing in single enzyme scenarios, and were able to examine the nonlinearity of drug-drug interactions. However, a universally-applicable platform that operates within the framework of systems-level response and simultaneously addresses the linkage between input stimuli and phenotypic variation across the cellular, tissue, and organism level has not been realized. To address this need, the FSC platform is developed to systematically utilize various stages to achieve globally optimized combinatorial design. Based on experimental data obtained via FSC, a drug efficacy-dose response was discovered to be represented by smooth quadratic surfaces. In this example, the use of the FSC platform has demonstrated that this smooth surface can be obtained without iterative feedback searches and can be rapidly interrogated to identify a global optimum for efficacy and safety with rapid convergence rate.

The use of FSC to optimize combination nanomedicine drug delivery in this example can be described in 3 steps (FIG. 3). Step 1 includes the design of the input stimuli, or drug-dosing regimen. Step 2 is based on the experimental screening of the phenotypic response of the biological system being interrogated, and step 3 involves an optimization technique-assisted search and convergence towards a global optimum. FSC reconciles experimental findings to construct a phenotypic response profile, map, or surface to pinpoint the global maximum in a deterministic fashion. In step 1, various ways of selecting drug—dose combinations can be used. Here, Latin hypercube sampling is used, which allowed a wide coverage of the concentration domain, to include diversified drug combinations for initial response surface construction. In step 2, the efficacy and safety of these combinations were assessed in 3 breast cancer cell lines of varying resistance profiles (MDA-MB-231, MCF7, and BT20), and 3 control cell lines (Breast fibroblast: MCF10A, Lung fibroblast: IMR-90, and Cardiomyocyte: H9C2) using viability assays to construct therapeutic windows. In step 3, a response map was then constructed by regression analysis using a tailored dose response model. The differential evolution optimization technique subsequently converged upon a global optimum drug combination within a single experiment (FIG. 3).

While FSC is a broadly applicable platform that can be applied towards both unmodified drugs and feasibly all nanoparticle carriers, nanodiamonds (NDs) were utilized as the drug delivery vehicles for this example and were compared against systematically optimized unmodified drug combinations to demonstrate the broad applicability of FSC. NDs represent promising platforms for cancer therapy since they have markedly improved the efficacy and safety of treatment in multiple pre-clinical studies. Furthermore, they are scalable materials and capable of carrying a broad spectrum of compounds. Optimal combinations comprised of three nanodiamond (ND)-drug combinations including ND-Doxorubicin (ND-DOX), ND-Mitoxantrone (ND-MTX), and ND-Bleomycin (ND-BLEO) as well as unmodified paclitaxel (PAC) were shown to outperform randomly formulated combinations, combinations of unmodified compounds, as well as both nanodiamond-modified and unmodified single drug regimens. Rather than account for all of the signaling pathway behaviors and genotypic properties of the biological system being addressed, the approach reported here rationally reconciled biological phenotypic response to therapeutic perturbation. Using this platform, complex modeling or theoretical assumptions are not relied to predict a treatment outcome. Rather, experimental validation of cellular outcomes from drug intervention was directly used to deterministically converge upon a multi-parametric, optimum drug combination. Therefore, the FSC platform serves as an efficient route towards globally optimal combination drug development.

Results and Discussion

Nanodiamond Drug Synthesis

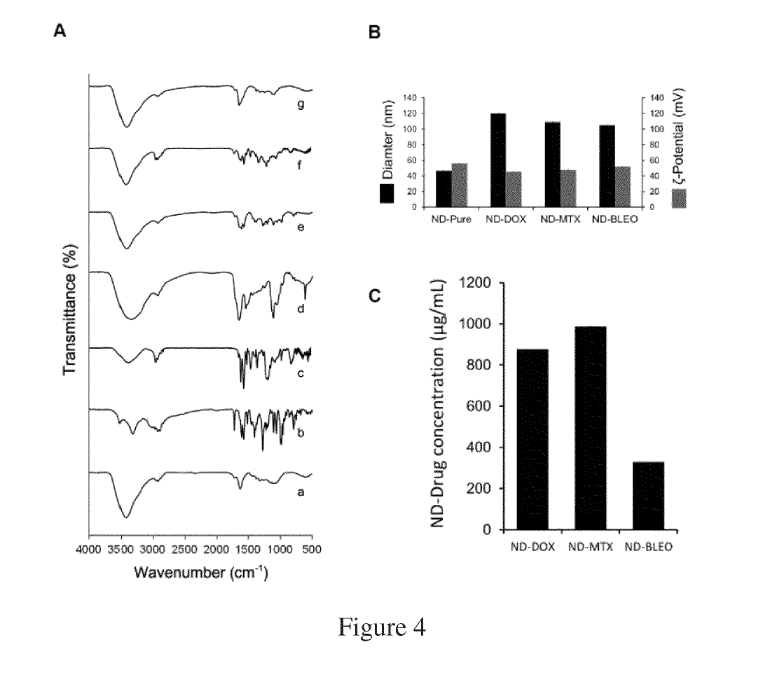

In order to confirm the presence of DOX, MTX, and BLEO on the NDs, Fourier transform infrared spectroscopy (FTIR) spectra were assessed by comparing the peaks of NDs, unmodified drugs, and ND-drugs (FIG. 4A). In the spectral region of 1700 cm⁻¹to 1800 cm⁻¹, all ND-drugs contained broad stretching vibrations of C═O from various carbonyl species formed on the ND surfaces, such as ketone, ester, lactone, and carboxylic acid. The peak at 1632 cm⁻¹ represented the bending vibration of O—H from adsorbed water on the NDs. In addition, the vibrational spectra observed with each unmodified drug and ND-drug complexes showed similar profiles overall and substantially identical vibrations in specific bands related to key functional groups of each drug molecules. In addition, vibration bands at 780˜850 cm⁻¹, 1560˜1590 cm⁻¹, 1603˜1616 cm⁻¹, and 1620˜1650 cm⁻¹that were visible from the ND-drug samples were noticeably absent in the ND-only samples. These peaks are indicative of C═C—H out-of-plain bending vibrations, two peaks representing C═C stretching vibrations, and C═O stretching vibrations, respectively. Due to the interaction between the NDs with benzene double bonds and other π bonds, the C═O stretching vibration was observed at a lower wavenumber than the usual C═O stretching vibration.

Particle size comparisons between drug-loaded and unmodified NDs were performed by dynamic light scattering (DLS) analysis. The hydrodynamic diameter of NDs in water was 46.6±0.17 nm. (FIG. 4B) Following drug adsorption, the hydrodynamic diameters of ND-DOX, ND-MTX, and ND-BLEO were increased to 120.0±0.93 nm, 109.2±0.58 nm, and 105.1±0.53 nm, respectively, confirming ND-drug interaction. Additionally, zeta potential measurements were performed (FIG. 4B). While the zeta potential of NDs was observed to be 55.8±0.37 mV, the zeta potentials of ND-drug complexes were slightly reduced to 45.3±0.51 mV (ND-DOX), 47.8±0.66 mV (ND-MTX), and 52.0±0.35 mV (ND-BLEO). Conclusively, both ND and ND-drugs show a narrow size distribution and similar zeta potentials in each sample, which indicate good homogeneity of particles in media. These are important properties in a scalable and translationally-relevant drug delivery system.

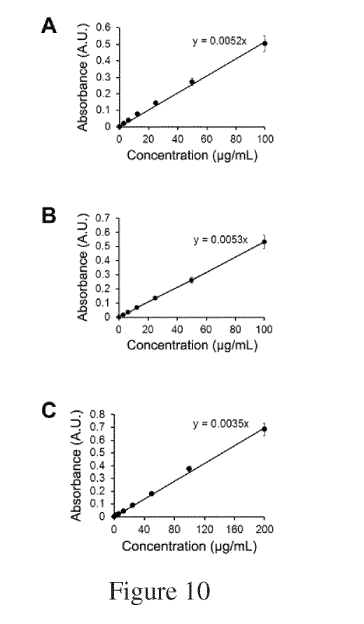

To determine the amount of drugs conjugated, UV spectroscopy was studied as described in Materials and Methods (FIG. 4C). The absorbance of the supernatants was measured at 550 nm for DOX, 590 nm for MTX, and 290 nm for BLEO, and the concentrations of drugs bound to the NDs are calculated via a standard curve derived by specific serial concentrations of drugs (FIG. 10). The loading efficiency of ND-DOX, ND-MTX, and ND-BLEO were 876±2.8 μg, 987±1.0 μg, and 329±1.0 μg, respectively.

Feedback System Control Optimization of Nanodiamond Combinations

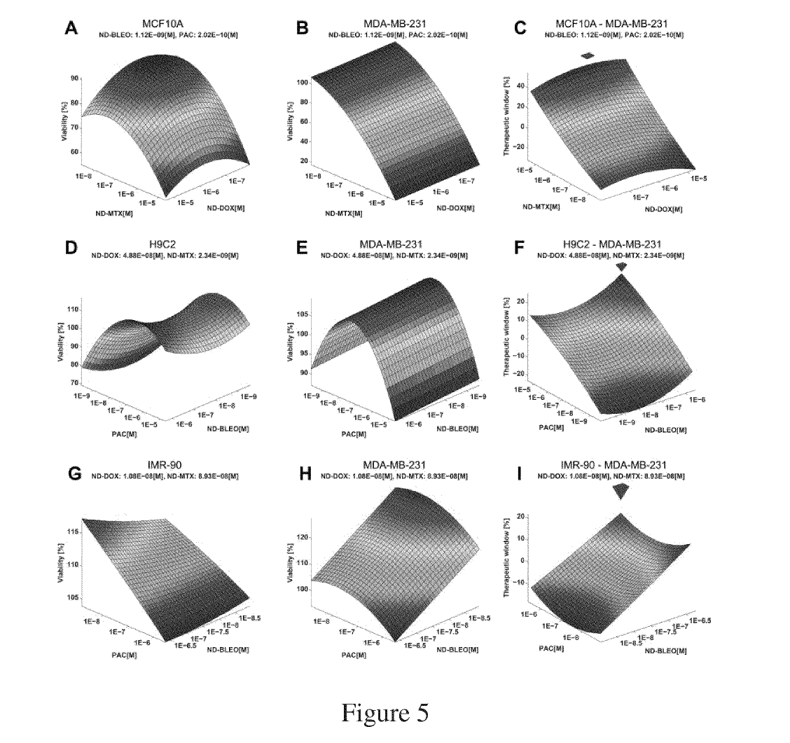

The drug combination utilized for this example was based on a panel of four drugs, including three that were ND-modified: ND-DOX, ND-BLEO, and ND-MTX, as well as one unmodified drug: PAC. The dose-response curves of the panel of drugs were constructed by conducting a 9-stage 2.5 fold serial dilution on a panel of 6 cell lines, including 3 cancer cell lines of varying levels of drug resistance, MDA-MB-231, BT20, and MCF7 and 3 control cells, MCF10A, H9C2, and IMR-90. The dose-response curves were applied to determine the concentration domain of each drug in each cell which resided between the maximum applicable concentration and the no response concentration (cell death <5%). For each drug, the final concentration domain was determined so that it covered the concentration domain of all cell lines. The concentration of each drug was discretized for FSC into 7 dilution stages in log scale to cover the specified range (Table 2). Latin hypercube sampling generated 57 drug combinations of different four drug concentrations from the panel of drugs (Table 3). The number of combinations was chosen to be 57 to provide definitive convergence to global optimal for in vitro and pre-clinical experimental validation, and ensure it exceeds the minimum specified sample size for multiple regression analysis to achieve desired statistical power given the anticipated effect size. The combinations were then added to the cell lines by a customized liquid handling robotic procedure in a high-throughput format, and the viabilities of the cells were measured by resazurin assay. The 5 dimensional cellular response surfaces were constructed with the experimental viability data sampled by Latin hypercube (FIG. 5).

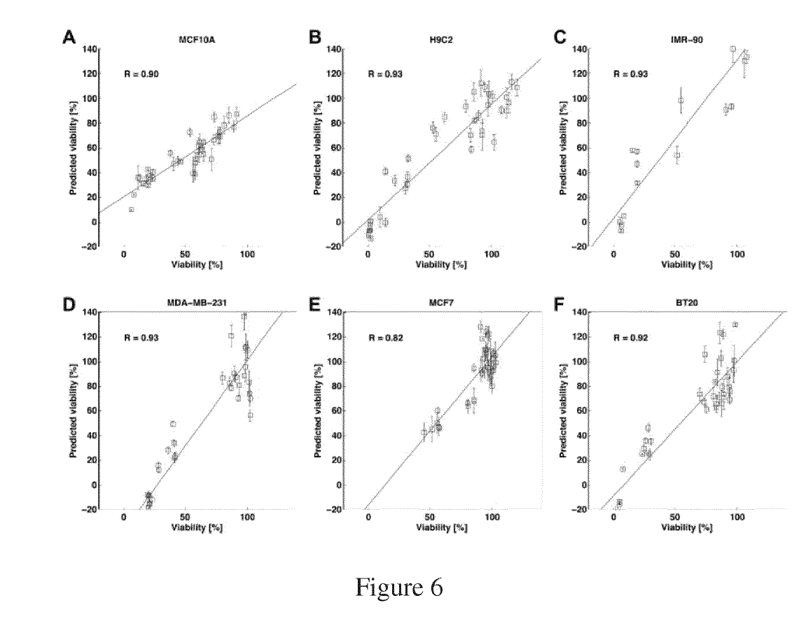

The response surfaces and therapeutic window surfaces are based on sections of 5 dimensional surfaces (FIG. 5A-I), from which two drugs are fixed as an anchor point, and the surface plotted by varying the concentrations of the two other drugs. The differential evolution optimization technique was applied onto the 5 dimensional surface of the therapeutic window to locate the global optimal dosage of the cell pair comprised of one cancer cell and one healthy cell. The efficacy of the identified optimum is experimentally verified. The quality of the response surfaces was ensured by including randomized combination samples in the verification experiments. To compare the FSC therapeutic window prediction with experimental verification, a t-test with the hypothesis that the experimental window was derived from normal distribution (with the mean as the prediction and unknown variance) was conducted. Therefore the null hypothesis will pass if the prediction and experimental values matched. The pyramid denotes the experimentally-verified optimal therapeutic window for MCF10A and MDA-MB-231 (FIG. 5C), which was shown to be 51.50±3.51%, with the bottom tip of the pyramid representing the experimental mean, and the height of the pyramid representing the standard deviation. The specific drug doses that comprised the optimum ND combination were ND-DOX: 9.88E-7 M, ND-BLEO: 1.12E-9 M, ND-MTX: 2.10E-5 M, and PAC: 2.02E-10 M. Of note, the FSC-prescribed optimum therapeutic window was located at the same concentration as the experimentally-verified dose represented by the pyramid and had a value of 47.01%. The case of H9C2 versus MDA-MB-231 and IMR-90 versus MDA-MB-231 are shown in FIGS. 5F and 5I, respectively. The experimental optimal therapeutic window was shown to be close to the FSC prediction with high p-values (0.78 and 0.47 respectively). The FSC predictions were compared with verification results, and a Pearson correlation was determined to assess the accuracy (FIG. 6). Importantly, in each experimental condition, it was evident that FSC can effectively and accurately predict the result and converge on an experimentally-verified optimum.

Feedback System Control-Optimized Nanodiamond Combinations Outperform Single Drugs

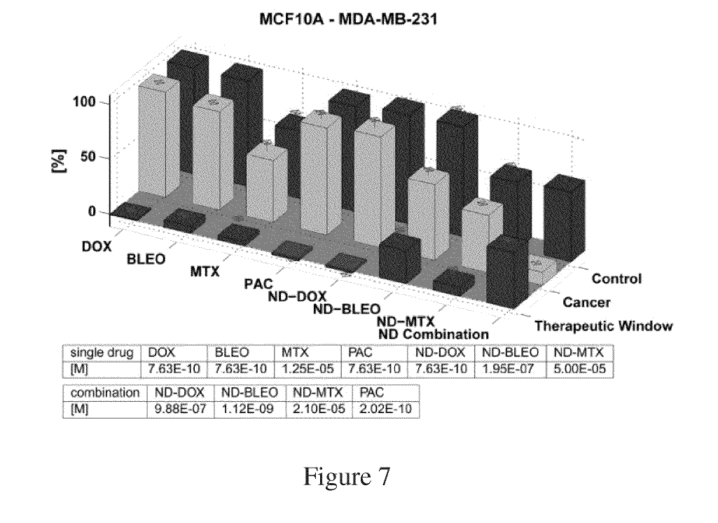

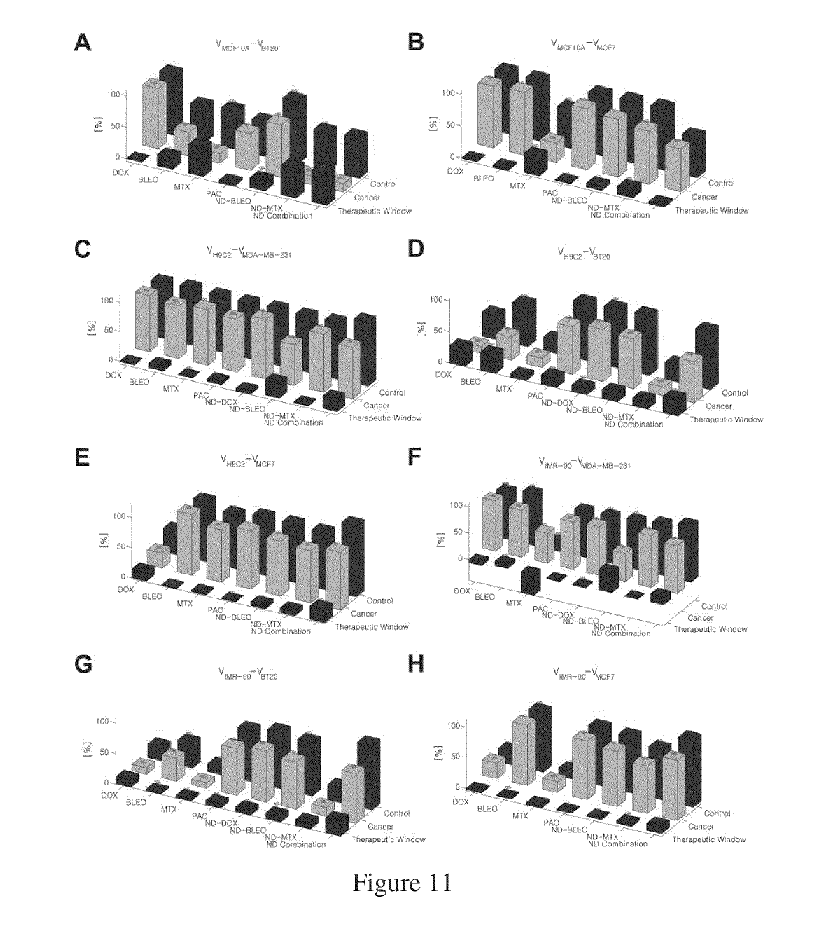

In order to confirm that the FSC-optimized drug combinations outperform single drug administration, the performance, which was measured by therapeutic window, of single drugs and the ND combination drugs were experimentally determined on a panel of 6 cell lines, including cancer cell lines, MDA-MB-231, MCF7, and BT20, and 3 control cell lines, MCF10A, H9C2, and IMR-90 (FIG. 7 and FIG. 11). Each individually administered drug, both unmodified and ND-modified, was serially diluted and applied to the panel of 6 cell lines; the optimal dosages, which generated the highest therapeutic window in each cancer and control pair, were determined by the optimal experimental result. The optimal ND combination for each cancer and control pair was prescribed by FSC and experimentally verified. Given that the dosing of all possible drug combinations encompassed that of the single drug, the therapeutic window of the global optimal combination was expected to outperform all single drugs.

In comparing the FSC-optimized ND combination with optimal single drug administration, it was observed that the ND combination outperforms all of the single drugs as measured by the therapeutic window of MCF10A and MDA-MB-231 (FIG. 7). The optimal ND combination-mediated therapeutic window was 51.50±3.51%, and the best single drug-mediated therapeutic window was 30.22±10.62% via ND-BLEO. Therefore, the optimal ND combination outperformed the best single drug by 21.28%. Interestingly, the main effector compounds in the optimal ND combination were ND-DOX and ND-MTX, but not ND-BLEO which was the best performing single drug. In addition, it could be seen that the rational design of an ND-modified drug combination resulted in markedly different drug concentrations from the single drugs (Table in FIG. 7).

FSC Optimized Nanodiamond Combinations Outperform Optimal Unmodified Combination Drugs

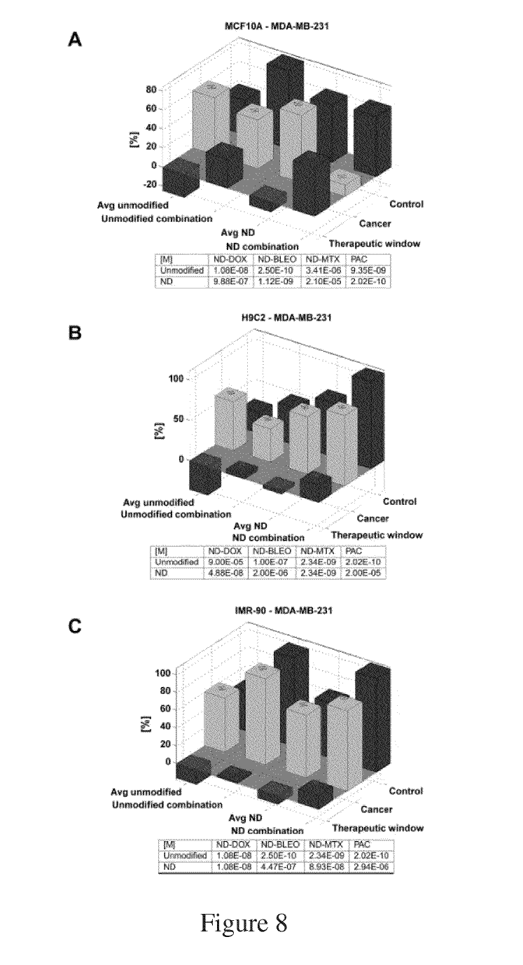

In addition to comparing ND-modified drug performance against single drug-mediated therapeutic windows, a comparative study between FSC-optimized ND-drug combinations and unmodified drug combinations was performed. The optimal ND-modified and unmodified combinations of DOX, BLEO, MTX and PAC were determined via FSC on a panel of 4 cell types including one cancer cell line, MDA-MB-231, and 3 control cell lines, MCF10A, H9C2, and IMR-90 (FIG. 8). To evaluate whether optimal ND combination outperforms that of unmodified combination, testing was performed on the null hypothesis that the difference of two therapeutic windows came from normal distribution with zero mean and unknown variance, using the paired-sample t-test. In the case of MCF10A versus MDA-MB-231, the therapeutic window of the optimal ND combination and unmodified combination were 51.50±3.51% and 31.10±2.50% respectively. Therefore, the optimal ND combination therapeutic window outperformed the optimal unmodified combination by 20.40% (p=0.0146, FIG. 8A). In the case of H9C2 versus MDA-MB-231, the therapeutic window of the optimal ND combination and unmodified combination was 21.46±4.00% and 4.94±5.06% respectively. Therefore, the optimal ND combination outperformed the optimal unmodified combination by 16.52% (p=0.0012, FIG. 8B). In the case of IMR-90 versus MDAMB-231, the therapeutic window of the optimal ND combination and unmodified combination was 15.80±6.19% and 2.20±3.66%, respectively. In this scenario, the optimal ND combination outperformed the optimal unmodified combination by 13.6% (p=0.0301, FIG. 8C). FIG. 8A-C indicated that the FSC-prescribed ND-drug combinations were comprised of markedly different drug doses towards the mediating of rationally improved therapeutic windows. Importantly, these values demonstrate that FSC can converge on specific drug ratios that mediate these optimal responses. ND-modified drugs can overcome issues such as drug resistance, or improve intratumoral retention, among other benefits which likely serve as foundations for the improved efficacy of ND-modified drug combinations over unmodified combinations.

FSC Optimized Nanodiamond Combinations Outperform Randomly Sampled ND Drug Combinations

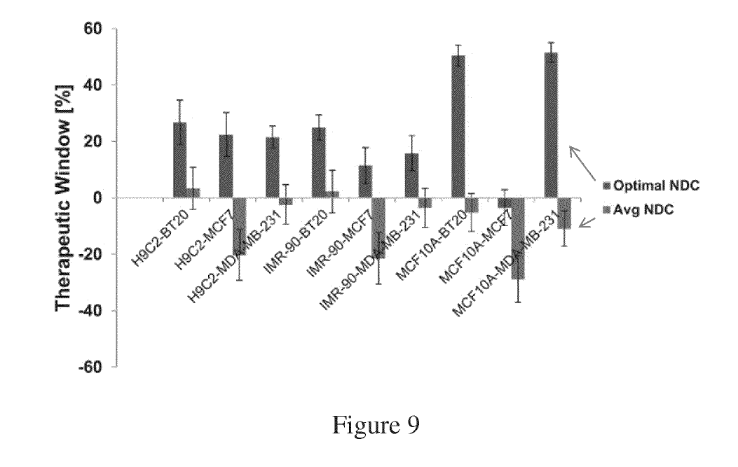

Additive or dose escalation-designed drug combinations are current clinical standards that are virtually precluded from mediating optimal therapeutic efficacy and safety. Given the sheer magnitude of the entire therapeutic search, it is unlikely that a randomized sampling method can accurately locate the global optimum. A comparison was made of the performance (therapeutic window) of randomly sampled ND-drug combinations with that of an FSC-optimized ND combination, and showed that randomized combinations usually lead to suboptimal results. Specifically, the average experimental therapeutic window was taken of the 57 randomized combinations obtained via Latin hypercube sampling, and this value was compared with the experimental optimal combination determined by FSC. (FIG. 9) In the case of H9C2 versus MCF7, the optimal dosage outperformed the average significantly by 42.66% (p=6.39E-04, Table 4). In the most extreme case, MCF10A versus MDA-MB-231, the optimal ND combination outperformed the average combination by 62.43% (p=9.39E-18, Table 4). Furthermore, among the 57 random combinations sampled, just 22.8% of the samples mediated a positive therapeutic window. It should also be noted that the application of random sampling methods such Gaussian distribution or uniform distribution over an FSC-prescribed 57 combination search would have resulted in an even lower percentage of samples exhibiting a positive therapeutic window. Provided that a random sampling method samples the space uniformly, the likelihood of consistently finding a combination with a positive therapeutic window is highly challenging, and therefore indicates that a systematic and rational platform should be utilized to provide convergence towards a global optimum.

Reconciling Phenotypic Information to Optimize Nanodiamond-Drug Combinations

In this example, DOX, MTX, and BLEO were selected because they bind potently and rapidly to NDs. In addition the ND-drug interaction has been shown to markedly reduce toxicity while the drug is being carried. Specifically, the ND-drugs provide markedly improved drug tolerance when carried such that treatment toxicity was reduced or eliminated via ND-binding. Therefore, the ND served as a scalable delivery agent for rapid ND-modified drug synthesis that simultaneously improved drug safety and tolerance. Unmodified PAC was utilized to demonstrate the modular nature of FSC and its ability to deliver a combination of both modified and unmodified therapeutic agents. While the ND-modified drugs used in this example were similar in structure and in function, they served as model chemotherapeutic agents, and the ND carrier and FSC platform are capable of being adapted towards virtually any therapeutic compounds for other indications as well.

A phenotype (e.g., viability) of a diseased biological complex system (e.g., a cell, an animal, or a human) under drug treatment can be expressed as a function, V(s, x), of the disease causing mechanisms, s, and the drug concentrations, x. According to the Taylor expansion in mathematics, V(s, x) can be related to the diseased biological complex system before therapeutic intervention, V(s, 0), as:

$$V ( s , x ) = V ( s , 0 ) + {\sum }_{k} {a}_{k} {x}_{k} + {\sum }_{l} {b}_{l} {x}_{l}^{2} + {\sum }_{m} {\sum }_{n} {c}_{mn} {x}_{m} {x}_{n} + high order terms$$

It has been found that the higher order terms are much smaller than the first and second order terms. Thus, the efficacy of the combinatorial drugs can be expressed as

$$V ( s , x ) - V ( s , 0 ) \approx {\sum }_{k} {a}_{k} {x}_{k} + {\sum }_{l} {b}_{l} {x}_{l}^{2} + {\sum }_{m} {\sum }_{n} {c}_{mn} {x}_{m} {x}_{n}$$

Due to the complexities of the disease causing mechanisms in the genome and in protein networks, the explicit functions of V(s, x) and V(s, 0) for the diseases are unknowns. However the efficacy, namely the differences of the two functions, can be expressed by a quadratic algebraic series. A small number of tests in various dose combinations can determine the coefficients of the algebraic series and locate the optimal dose combination from a very large combinatorial drug-dose parameter space. In other words, the FSC technique can bypass the identification of the disease causing mechanisms and converge on the optimal treatment of achieving the multi-dimensional desired endpoint outputs.

The fitted response surfaces of two selected cancer and control cell pairs were superimposed to form a 5 dimensional surface of the therapeutic window h(x), where h(x) is specified as:

h(x)=FSC_{control}(x)−FSC_{cancer}(x)

where FSC_{control}(x)and FSC_{cancer}(x) is the viability for the control cell and the cancer cell, respectively. Indeed, a powerful feature of FSC is the ability to be able to superimpose multiple response surfaces to achieve simultaneous multi-objective optimization. h is specified as the superposition of two cells in this example for simplicity of interpretation as well as proof of principle.

This example has highlighted that, due to synergistic and antagonistic effects between different drugs, therapeutic efficacy cannot be readily determined by multiple dose-response curves of single drugs. Instead, the therapeutic window is fully represented by a multi-dimensional response surface. The optimal dosage or combination of drugs that can maximize the therapeutic window is located within this multi-dimensional response surface. For instance, a N-drug combination with M different concentrations will result in M^{N} possible combinations. Conventional methods (e.g., High throughput screening) is a measure-all approach, which is inefficient because the possible number of drug combinations grows exponentially with each increase in the number of drugs that comprise the combination. Furthermore, the measure-all approach cannot be extended to in vivo studies or clinical trials. FSC has thus demonstrated a particular advantage in this case, resolving the complete response surface with a small number of experiments while rapidly converging on the global optimum.

Multi-Parametric Optimization of Nanodiamond Drug Combinations via Feedback System Control

Following the demonstration that FSC-optimized ND combinations outperform single drugs in terms of efficacy and safety, a finding was that most of the single drugs did not mediate a large therapeutic window. For example, DOX, BLEO, MTX, and ND-MTX exhibited therapeutic windows of less than 10% and thus did not have a major therapeutic effect. Single drug administration still serves as a clinical standard of treatment for several cancers, and innate or acquired drug resistance and a decline in efficacy, as well as toxicity, remain major obstacles to single drug administration. To overcome these issues, nanomedicine has been used to overcome resistance and reduce toxicity in pre-clinical and now clinical studies. Since combination therapy is a widely adopted strategy to even further enhance treatment efficacy, this finding confirms the use of combination nanomedicine to improve treatment efficacy over single unmodified drug or single modified drug administration.

The ability to rationally design and experimentally verify a globally-optimized ND-modified drug combination from this example also highlights the ability for FSC to systematically address the issue of resistance in cancer. The selected BT20, MCF7, and MDAMB-231 cancer cell lines each possess varying drug resistance profiles, and the ability for the FSC platform to rapidly converge upon an optimum combination that accounts for this resistance thus allows for a dynamic response to the cancer cells' ability to evolve against drug-induced cytotoxicity. Therefore, in a scenario where a drug, either unmodified or ND-modified, is rendered inactive against a particular type of cancer, this drug can be removed from a combinatorial design and replaced with a new candidate compound. A subsequent re-designed, globally optimized combination can be then prescribed and verified for rapid implementation. Importantly, it should be also noted that following the in vitro selection of FSC-optimized drug combination candidates as shown in this example, downstream pre-clinical and translational optimization is capable of using additional FSC-derived drug design platforms that can multi-parametrically maximize safety and efficacy.

Material and Methods

Synthesis and Characterization of Nanodiamond-Modified Drugs

NDs were obtained from the NanoCarbon Research Institute Ltd. (Nagano, Japan). Doxorubicin hydrochloride and mitoxantrone dihydrochloride were purchased from Sigma-Aldrich (Milwaukee, USA), and bleomycin sulfate was acquired from Cayman chemical (Ann Arbor, USA). All samples and solvents were autoclaved prior to use. To formulate the ND-drug complexes, the drugs were mixed with sterilized deionized water at 5 mg/mL. Autoclaved NDs were then mixed at a ratio of 5:1 (w/w) with doxorubicin and mitoxantrone and 5:2 (w/w) with bleomycin, followed by the addition of NaOH for coupling drugs onto the NDs to a final NaOH concentration of 2.5 mM. The ND-drugs were mixed thoroughly, and incubated for 4 days at room temperature. Subsequently, the ND-drug suspensions were centrifuged, and washed with deionized water until transparent ND-drug solutions were obtained. The final products were re-suspended in water with concentrations of 5 mg/mL (NDs/water, w/v) for loading efficiency and nanoparticle characterization.

During the washing/centrifuging steps, supernatants containing unbound drugs were analyzed to determine the drug loading efficiencies. The absorbance of the supernatant containing free DOX, MTX, and BLEO was measured at 550 nm, 590 nm, and 290 nm, respectively. The concentration of drug incorporated onto the NDs was calculated via standard curves which were set by the absorbance values of serial dilutions of the drugs within a range from 0 to 200 μg/mL.

To confirm drug presence on the NDs, FTIR spectroscopy was performed (Jasco FT/IR-420). Prior to FTIR analysis, 2 mg of ND, drugs, and ND-drug samples were mixed with 100 mg of potassium bromide (KBr) by using mortar and pestle, and then pelletized to make thin discs for further analyses with the resolution of 1 cm⁻¹and 64 scan accumulations.

The size and zeta (ζ) potential of NDs and ND-drug complexes (0.2-0.3 mg/mL) were measured using a Zetasizer Nano ZS (Malvern Instrument, UK). Nanoparticle sizes were measured at a 173° backscattering angle with at least 3 runs at 25° C. The hydrodynamic diameter was determined from the z-average values from runs in triplicate. Zeta potential was also determined at 25° C. in water by using DTS-1060C clear zeta cells in automatic mode.

Cell Culture and Plating

MCF10A (human epithelial breast cells), H9C2 (rat myocardium myoblast cells), IMR-90 (human lung fibroblast cells), BT20 (human breast carcinoma cells), MCF7 (human breast carcinoma cells), and MDA-MB-231 (human breast adenocarcinoma cells) were obtained from American Type Culture Collection (ATCC) and were cultured according to manufacturer protocols. Cells were subsequently detached via trypsin-EDTA, counted via hemocytometer (Hausser), and seeded into 96-well plates via Biomek FXp (Beckman Coulter).

Single Drug and Drug Combination Evaluation

For the single drug study, unmodified and ND-modified doxorubicin, bleomycin and mitoxantrone were used. Paclitaxel was used in the unmodified version. Serial dilutions of the drugs were made on a 96-well plate and then applied to the corresponding wells on a cell plate. These steps are repeated for the remaining cell lines. For the drug combination study, concentrations of drugs were determined using the outcome of the single drug experiments. ND drugs or unmodified drugs were applied onto a 48-well plate, and serial dilutions were made. Drug combinations, according to Latin hypercube sampling, were then made on a 96-well plate. These drug solutions were then transferred to the corresponding wells on a cell plate. These procedures were applied for all cell plates. Cells were incubated in drug solutions at 37° C. for 72 hours.

Cell Viability Assays

To determine cell viability, 0.5 mM resazurin (Sigma) was applied to the 96-well cell plates, followed by an incubation period of 3 hours at 37° C., and 5% CO₂. Cell viability was measured by fluorescence readings at 560 nm/590 nm.

Latin Hypercube Sampling

The drug concentration range was determined by single drug-dose response experiments of the cancer model to be between the maximum achievable concentration and the zero effect concentration. The log concentration was taken, and the log concentration range of each drug was discretized into 7 stratums. A Latin hypercube sampling process was performed on the sample space, ensuring that all subspaces were comprehensively covered. Following objective function determination in order to account for maximal therapeutic efficacy against the cancer cell lines while minimizing toxicity against control cell lines, differential evolution was applied to rapidly identify globally optimized drug combinations for subsequent verification.

Latin hypercube sampling was performed upon the sample space where the input X∈Ω. Let the sample be Xᵢⱼ, i=1, . . . , N and j=1, . . . , K. The range of Xᵢ is discretized into N strata, and a component from each stratum was selected. The components were rounded up to the closest digits and treated as stages in the log concentration range. Each sampled component was randomly assigned into the final design matrix X. In this way, Ω was divided into N^{K} cells, which represented a hypercube discretization of the sample space. The Latin hypercube sampling process ensured that all subspaces of Ω were fully covered, and the components are sampled in a stratified manner.

Statistical Analysis

A student t-test was utilized for statistical analysis of FSC-based optimization of ND-drug combinations. Analysis was completed for studies comparing FSC-optimized ND-modified combinations with modified and unmodified single drugs, unmodified drug combinations, as well as randomly-sampled ND-modified combinations. A p-value of less than 0.05 was deemed to represent statistical significance in these cases. For the response surface formulation studies, a t-test with the hypothesis that the experimental window was derived from normal distribution with the mean as the prediction and unknown variance was conducted. Therefore the null hypothesis was considered to pass if the prediction and experimental values matched. Pearson correlation analysis was also plotted to further correlate FSC predictions with experimental verification.

TABLE 2
Maximum concentration of ND drugs in ND
combination and fold of serial dilution.

| name | max concentration [M] | fold |
|---|---|---|
| ND-DOX | 9.00E−05 | 4.50 |
| ND-BLEO | 2.00E−06 | 4.47 |
| ND-MTX | 1.30E−04 | 6.18 |
| PAC | 2.00E−05 | 6.80 |

TABLE 3
Latin hypercube sampling of drug combinations. Stage
is the stage number in a 7-stage serial dilution.

[stage]

| # | ND-DOX | ND-BLEO | ND-MTX | PAC |
|---|---|---|---|---|
| 1 | 3 | 1 | 4 | 1 |
| 2 | 4 | 2 | 5 | 3 |
| 3 | 2 | 3 | 5 | 4 |
| 4 | 1 | 5 | 5 | 5 |
| 5 | 4 | 6 | 3 | 7 |
| 6 | 7 | 3 | 5 | 5 |
| 7 | 2 | 7 | 3 | 3 |
| 8 | 2 | 1 | 6 | 7 |
| 9 | 2 | 5 | 1 | 6 |
| 10 | 2 | 4 | 6 | 4 |
| 11 | 5 | 7 | 2 | 5 |
| 12 | 4 | 6 | 5 | 4 |
| 13 | 4 | 2 | 3 | 7 |
| 14 | 5 | 3 | 4 | 3 |
| 15 | 6 | 2 | 2 | 6 |
| 16 | 2 | 3 | 3 | 6 |
| 17 | 3 | 3 | 5 | 3 |
| 18 | 1 | 3 | 7 | 5 |
| 19 | 2 | 6 | 6 | 7 |
| 20 | 1 | 4 | 4 | 6 |
| 21 | 2 | 5 | 7 | 2 |
| 22 | 6 | 4 | 4 | 1 |
| 23 | 6 | 5 | 5 | 4 |
| 24 | 4 | 2 | 6 | 1 |
| 25 | 6 | 5 | 2 | 2 |
| 26 | 3 | 3 | 1 | 1 |
| 27 | 4 | 1 | 6 | 5 |
| 28 | 3 | 6 | 3 | 2 |
| 29 | 5 | 6 | 5 | 7 |
| 30 | 7 | 2 | 7 | 5 |
| 31 | 3 | 4 | 2 | 6 |
| 32 | 5 | 5 | 2 | 3 |
| 33 | 4 | 2 | 4 | 3 |
| 34 | 5 | 1 | 4 | 5 |
| 35 | 6 | 3 | 4 | 6 |
| 36 | 3 | 2 | 4 | 6 |
| 37 | 7 | 6 | 2 | 6 |
| 38 | 1 | 6 | 3 | 6 |
| 39 | 4 | 6 | 3 | 4 |
| 40 | 6 | 7 | 1 | 4 |
| 41 | 2 | 4 | 2 | 2 |
| 42 | 3 | 5 | 7 | 4 |
| 43 | 3 | 3 | 6 | 5 |
| 44 | 4 | 2 | 5 | 2 |
| 45 | 6 | 3 | 1 | 3 |
| 46 | 3 | 4 | 4 | 3 |
| 47 | 5 | 4 | 1 | 4 |
| 48 | 7 | 5 | 4 | 4 |
| 49 | 6 | 4 | 6 | 3 |
| 50 | 6 | 6 | 6 | 1 |
| 51 | 7 | 4 | 2 | 3 |
| 52 | 2 | 6 | 2 | 2 |
| 53 | 5 | 2 | 5 | 5 |
| 54 | 5 | 5 | 3 | 5 |
| 55 | 6 | 7 | 3 | 2 |
| 56 | 5 | 2 | 6 | 2 |
| 57 | 3 | 6 | 7 | 2 |

TABLE 4
Optimal therapeutic window versus average therapeutic window (n = 3).

| | | H9C2- | H9C2- | H9C2- | IMR-90- | IMR-90- | IMR-90- |
|---|---|---|---|---|---|---|---|
| | Cell pairs | BT20 | MCF-7 | MDA0MB-231 | BT20 | MCF7 | MDA-MB-231 |
| [%] | OPT TW | 0.27 | 0.22 | 0.21 | 0.25 | 0.11 | 0.16 |
| | STD | 0.08 | 0.08 | 0.04 | 0.04 | 0.06 | 0.06 |
| | AVG TW | 0.03 | −0.20 | −0.02 | 0.02 | −0.21 | −0.04 |
| | AVG STD | 0.07 | 0.09 | 0.07 | 0.08 | 0.09 | 0.07 |
| concentration | ND-DOX | 9.88E−07 | 4.88E−08 | 4.88E−08 | 2.19E−07 | 9.88E−07 | 1.08E−08 |
| | ND-BLEO | 1.12E−09 | 2.00E−06 | 2.00E−06 | 2.24E−08 | 1.12E−09 | 4.47E−07 |
| | ND-MTX | 3.41E−06 | 8.93E−08 | 2.34E−09 | 1.45E−08 | 5.52E−07 | 8.93E−08 |
| | PAC | 9.35E−09 | 9.35E−09 | 2.00E−05 | 2.94E−06 | 9.35E−09 | 2.94E−06 |
| | p-value | 3.99E−02 | 6.39E−04 | 4.96E−03 | 1.35E−02 | 4.89E−03 | 4.53E−02 |

| | | MCF10A- | MCF10A- | MCF10A- |
|---|---|---|---|---|
| | Cell pairs | BT20 | MCF7 | MDA0MB-231 |
| [%] | OPT TW | 0.50 | −0.04 | 0.52 |
| | STD | 0.04 | 0.06 | 0.04 |
| | AVG TW | −0.05 | −0.29 | −0.11 |
| | AVG STD | 0.07 | 0.08 | 0.06 |
| concentration | ND-DOX | 9.88E−07 | 9.88E−07 | 9.88E−07 |
| | ND-BLEO | 1.12E−09 | 1.12E−09 | 1.12E−09 |
| | ND-MTX | 2.10E−05 | 2.10E−05 | 2.10E−05 |
| | PAC | 2.02E−10 | 2.02E−10 | 2.02E−10 |
| | p-value | 1.51E−12 | 2.11E−02 | 9.39E−18 |

As used herein, the singular terms “a,” “an,” and “the” include plural referents unless the context clearly dictates otherwise. Thus, for example, reference to an object can include multiple objects unless the context clearly dictates otherwise.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to less than or equal to ±5%, such as less than or equal to ±4%, less than or equal to ±3%, less than or equal to ±2%, less than or equal to ±1%, less than or equal to ±0.5%, less than or equal to ±0.1%, or less than or equal to ±0.05%.

While this disclosure has been described with reference to the specific embodiments thereof, it should be understood by those skilled in the art that various changes may be made and equivalents may be substituted without departing from the true spirit and scope of this disclosure as defined by the appended claims. In addition, many modifications may be made to adapt a particular situation, material, composition of matter, method, operation or operations, to the objective, spirit and scope of this disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of this disclosure. Accordingly, unless specifically indicated herein, the order and grouping of the operations is not a limitation of this disclosure.

What is claimed is:

1. A method, comprising:
specifying a cost function to optimize a combination of N drugs, the cost function including at least one phenotypic contribution corresponding to efficacy and at least one phenotypic contribution corresponding to safety, at least one of the N drugs is a nanomaterial-modified drug, with N being 2 or more;
conducting in vitro or in vivo tests by applying varying combinations of dosages of the N drugs to determine the phenotypic contributions from results of the tests;
fitting the results of the tests into a representation of the cost function; and
using the representation of the cost function, identifying at least one optimized combination of dosages of the N drugs.

2. The method of claim 1, wherein the cost function is a sum of the phenotypic contributions.

3. The method of claim 1, wherein the cost function is a quadratic function of dosages of the N drugs.

4. The method of claim 3, wherein the quadratic function includes m parameters, with m=1+2N+ (N(N−1))/2, and fitting the results of the tests includes deriving the m parameters.

5. The method of claim 1, wherein the representation of the cost function is a multi-dimensional surface, and identifying the at least one optimized combination of dosages of the N drugs includes identifying an extremum of the surface.

6. The method of claim 5, wherein identifying the extremum is carried out by applying a stochastic optimization technique.

7. The method of claim 5, wherein identifying the extremum is carried out by applying a deterministic optimization technique.

8. The method of claim 1, further comprising selecting the N drugs from a pool of P drugs, with P>N.

9. A method, comprising:
evaluating a pool of P drugs to identify multiple optimized subsets of the P drugs having respective values of a therapeutic outcome;
ranking the optimized subsets according to their respective values of the therapeutic outcome;
selecting an optimized subset from the ranked optimized subsets, the selected optimized subset being a combination of N drugs, with N<P;
modifying at least one of the N drugs with a nanomaterial to provide a nanomaterial-modified combination of the N drugs; and
evaluating the nanomaterial-modified combination of the N drugs to identify an optimized combination of dosages of the N drugs.

10. The method of claim 9, wherein the P drugs are unmodified drugs.

11. The method of claim 9, wherein P is 5 or more.

12. The method of claim 9, wherein P is 10 or more.

13. The method of claim 9, wherein evaluating the pool of P drugs includes:
conducting in vitro or in vivo tests by applying varying combinations of dosages of the P drugs;
fitting results of the tests into a multi-dimensional representation of the therapeutic outcome; and
using the representation of the therapeutic outcome, identifying the optimized subsets of the P drugs.

14. The method of claim 9, wherein evaluating the nanomaterial-modified combination of the N drugs includes:
conducting in vitro or in vivo tests by applying varying combinations of dosages of the N drugs;
fitting results of the tests into a multi-dimensional representation of the therapeutic outcome; and
using the representation of the therapeutic outcome, identifying the optimized combination of dosages of the N drugs.

15. The method of claim 14, wherein evaluating the nanomaterial-modified combination of the N drugs further includes specifying the therapeutic outcome as a low order function of dosages of the N drugs.

16. The method of claim 15, wherein the low order function is a quadratic function including m parameters, with m=1+2N+ (N(N−1))/2, and fitting the results of the tests includes deriving the m parameters.

17. The method of claim 14, wherein the representation of the therapeutic outcome is a multi-dimensional surface, and identifying the optimized combination of dosages of the N drugs includes identifying an extremum of the surface.
